(19) United States
(12) Patent Application Publication
(10) Pub. No.: US 2018/0353602 A1
(43) Pub. Date: Dec. 13, 2018

(54) COMBINATION OF HDAC INHIBITOR AND ANTI-PD-L1 ANTIBODY FOR TREATMENT OF CANCER

(71) Applicant: Syndax Pharmaceuticals, Inc., Waltham, MA (US)

(72) Inventors: Robert GOODENOW, San Clemente, CA (US); Peter ORDENTLICH, Lexington, MA (US)

(21) Appl. No.: 15/739,107

(22) PCT Filed: Jun. 28, 2016
(86) PCT No.: PCT/US2016/039906

§ 371 Date: Dec. 21, 2017

Related U.S. Application Data
(60) Provisional application No. 62/186,237, filed on Jun. 29, 2015.

(51) Int. Cl. A61K 39/395; A61K 31/4406; A61K 9/00; G01N 33/574

(52) U.S. Cl. CPC A61K 39/3955; A61K 31/4406; A61K 2039/545; G01N 33/57415; A61K 9/0019

(57) ABSTRACT

Described herein are methods for the treatment of breast cancer in a subject. In particular, methods are provided for the treatment of metastatic triple negative breast cancer with a combination of entinostat and an anti-PD-L1 antibody, such as MPDL3280A.

### CROSS-REFERENCE

This application is a national stage application of and claims priority to International Patent Application No. PCT/US2016/039906 filed Jun. 28, 2016, which in turn claims the benefit of U.S. Provisional Application No. 62/186,237 filed Jun. 29, 2015. The present application incorporates herein by reference the disclosures of each of the above-referenced applications in their entireties.

### SUMMARY OF THE INVENTION

In one embodiment, is described a method of treating cancer in a patient, wherein the method comprises, administering to a patient a combination comprising entinostat and an anti-PD-L1 antibody. In additional embodiments are described methods wherein the anti PD-L1 antibody is MPDL3280A.

In additional embodiments, are described methods, wherein the cancer is characterized by overexpression of PD-L1. In additional embodiments, are described methods, wherein the cancer is breast cancer. In additional embodiments, are described methods, wherein the breast cancer is metastatic breast cancer. In additional embodiments, are described methods, wherein the breast cancer is estrogen receptor (ER), progesterone receptor (PR), and Her-2 negative breast cancer. In additional embodiments, are described methods, wherein the estrogen receptor (ER), progesterone receptor (PR), and Her-2 negative breast cancer is triple negative breast cancer. In additional embodiments, are described methods, wherein the triple negative breast cancer is metastatic triple negative breast cancer.

In additional embodiments, are described methods, wherein the entinostat and anti-PD-L1 antibody are administered sequentially in either order or simultaneously. In additional embodiments, are described methods, wherein the entinostat and anti-PD-L1 antibody are administered sequentially in either order or simultaneously, during a treatment cycle of 21 days. In additional embodiments, are described methods, wherein the anti-PD-L1 antibody is administered by intravenous infusion. In additional embodiments, are described methods, wherein the anti-PD-L1 antibody is administered once every three weeks during the treatment cycle, at a dose of 1200 mg. In additional embodiments, are described methods, wherein the entinostat is administered periodically during the treatment cycle. In additional embodiments, are described methods, wherein the entinostat is administered once every week during the treatment cycle, at a dose of 3 mg. In additional embodiments, are described methods, wherein the entinostat is administered once every week during the treatment cycle, at a dose of 5 mg. In additional embodiments, are described methods, wherein the entinostat is administered once every two weeks during the treatment cycle, at a dose of 10 mg. In additional embodiments, are described methods, wherein the entinostat is administered first. In additional embodiments, are described methods, wherein the entinostat is administered weekly. In additional embodiments, are described methods, wherein the entinostat is administered every two weeks. In additional embodiments, are described methods, wherein the entinostat is administered every two weeks, at a dose of 10 mg. In additional embodiments, are described methods, wherein the entinostat and anti-PD-L1 antibody are administered simultaneously.

In one embodiment, is described a kit for treating metastatic triple negative breast cancer comprising a combination of entinostat and an anti-PD-L1 antibody. In an additional embodiment, is described a kit, wherein the anti-PD-L1 antibody is MPDL3280A.

In some embodiments is described a method of treating cancer in a patient in need thereof, the method comprising administering to the patient a combination comprising entinostat and MPDL3280A, wherein the cancer is metastatic triple negative breast cancer.

Provided herein in one embodiment is a method of treating cancer in a patient in need thereof, wherein the method comprises administering to the patient a combination therapy comprising entinostat and an anti-PD-L1 antibody, wherein the cancer is metastatic triple negative breast cancer and the anti-PD-L1 antibody is MPDL3280A. Another embodiment provides a method of treating a cancer in a patient in need thereof, wherein the method comprises, administering to the patient a combination consisting essentially of entinostat and MPDL3280A. In some embodiments, the cancer is metastatic triple negative breast cancer. In some embodiments, the entinostat is administered as a solid dosage form and the MPDL3280A is administered as an intravenous infusion. In one embodiment is provided a method of selecting a patient for a combination therapy comprising administering entinostat and an anti-PD-L1 antibody, the method comprising measuring PD-L1 expression in a tumor tissue sample obtained from the patient. In some embodiments, the method further comprises administering the combination therapy to the patient if tumor proportion score (TPS) for PD-L1 expression is between 1% and 50%. In some embodiments, the method further comprises administering the combination therapy to the patient if tumor proportion score (TPS) for PD-L1 expression is greater than or equal to 1%. In some embodiments, the method further comprises administering the combination therapy to the patient if tumor proportion score (TPS) for PD-L1 expression is greater than or equal to 49%. In some embodiments, the tumor proportion score is measure in a tumor tissue sample from a metastatic triple negative breast cancer.

### INCORPORATION BY REFERENCE

All publications, patents, and patent applications described in this specification are herein incorporated by reference to the same extent as if each individual publication, patent, or patent application was specifically and individually indicated to be incorporated by reference.

### DETAILED DESCRIPTION

Provided herein are methods of treating cancer based on the administration of an HDAC inhibitor and an anti PD-L1 antibody. The methods may further include treatments wherein the combination is supplemented with one or more therapeutic agents or therapies.

To facilitate understanding of the disclosure set forth herein, a number of terms are defined below.

As used herein, “abnormal cell growth,” refers to cell growth that is independent of normal regulatory mechanisms (e.g., loss of contact inhibition), including the abnormal growth of normal cells and the growth of abnormal cells.

“Neoplasia” as described herein, is an abnormal, unregulated and disorganized proliferation of cells that is distinguished from normal cells by autonomous growth and somatic mutations. As neoplastic cells grow and divide they pass on their genetic mutations and proliferative characteristics to progeny cells. A neoplasm, or tumor, is an accumulation of neoplastic cells. In some embodiments, the neoplasm can be benign or malignant.

“Metastasis,” as used herein, refers to the dissemination of tumor cells via lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

As discussed herein, “angiogenesis” is prominent in tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors such as renal cell carcinoma, hepatocellular carcinoma, and benign tumors such as acoustic neuroma, and neurofibroma. Angiogenesis has been associated with blood-born tumors such as leukemias. It is believed that angiogenesis plays a role in the abnormalities in the bone marrow that give rise to leukemia. Prevention of angiogenesis could halt the growth of cancerous tumors and the resultant damage to the subject due to the presence of the tumor.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

Cancer, tumors, tumor-related disorders, and neoplastic disease states are serious and often times life-threatening conditions. These diseases and disorders, which are characterized by rapidly-proliferating cell growth, continue to be the subject of research efforts directed toward the identification of therapeutic agents which are effective in the treatment thereof. Such agents prolong the survival of the patient, inhibit the rapidly-proliferating cell growth associated with the neoplasm, or effect a regression of the neoplasm.

HDAC inhibitors are an emerging class of therapeutic agents that promote differentiation and apoptosis in hematologic and solid malignancies through chromatin remodeling and gene expression regulation. Several HDAC inhibitors have been identified including benzamides (entinostat), short-chain fatty acids (i.e., Sodium phenylbutyrate); hydroxamic acids (i.e., suberoylanilide hydroxamic acid and thrichostatin A); cyclic tetrapeptides containing a 2-amino-8-oxo-9, 10-epoxy-decanoyl moiety (i.e., trapoxin A) and cyclic peptides without the 2-amino-8-oxo-9, 10-epoxy-decanoyl moiety (i.e., FK228). Entinostat is a benzamide HDAC inhibitor undergoing clinical investigation in multiple types of solid tumors and hematologic cancers. Entinostat is rapidly absorbed and has a half-life of about 100 hours and, importantly, changes in histone acetylation persist for several weeks following the administration of entinostat.

High expression of PD-1/PD-L1 on tumor cells has been found to correlate with poor prognosis and survival in various other solid tumor types. Without being bound by any theory it is contemplated that the PD-1/PD-L1 pathway plays a critical role in the tumor immune evasion and could be considered an attractive target for therapeutic intervention in several solid organ types.

Several PD-1 and PD-L1 antibodies are in clinical development. Overall, they have been reported to be well tolerated, with most not reaching dose-limiting toxicity in their phase I studies.

### Histone Deacetylase

The HDACs are a family including at least eighteen enzymes, grouped in three classes (Class I, II and III). Class I HDACs include, but are not limited to, HADCs 1, 2, 3, and 8. Class I HDACs can be found in the nucleus and are believed to be involved with transcriptional control repressors. Class II HDACs include, but are not limited to, HDACS 4, 5, 6, 7, and 9 and can be found in both the cytoplasm as well as the nucleus. Class III HDACs are believed to be NAD dependent proteins and include, but are not limited to, members of the Sirtuin family of proteins. Non-limiting examples of sirtuin proteins include SIRT1-7. As used herein, the term “selective HDAC” refers to an HDAC inhibitor that does not interact with all three HDAC classes.

### HDAC Inhibitors

HDAC inhibitors can be classified broadly into pan HDAC inhibitors and selective HDAC inhibitors. Although there is a large structural diversity of known HDAC inhibitors, they share common features: a part that interacts with the enzyme active site and a side-chain that sits inside the channel leading to the active site. This can be seen with the hydroxamates such as SAHA, where the hydroxamate group is believed to interact with the active site. In the case of the depsipeptides, it is believed that an intracellular reduction of the disulphide bond creates a free thiol group (which interacts with the active site) attached to a 4-carbon alkenyl chain. A difference between the HDAC inhibitors is in the way that they interact with the rim of the HDAC channel, which is at the opposite end of the channel to the active site. It is this interaction, between the HDAC inhibitor and the rim of the channel, which is believed to account, at least in part, for some observed differences in HDAC selectivity between pan-HDAC inhibitors, such as SAHA and selective HDAC inhibitors such as the depsipeptides. A particularly preferred HDAC inhibitor is entinostat. Entinostat has the chemical name N-(2-aminophenyl)-4-[N-(pyridine-3-yl)methoxycarbonylamino-methyl]-benzamide and the chemical structure shown below.

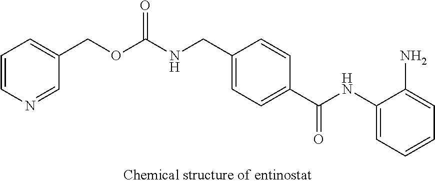 [SMILES: Nc1ccccc1NC(=O)c1ccc(CNC(=O)OCc2cccnc2)cc1]

### Programmed Cell Death-1 (PD-1)

PD-1 is a cell surface receptor that is a member of the CD28 family of T-cell regulators, within the immunoglobulin superfamily of receptors. The human PD-1 gene is located at chromosome 2q37, and the full-length PD-1 cDNA encodes a protein with 288 amino acid residues with 60% homology to murine PD-1. It is present on CD4−CD8− (double negative) thymocytes during thymic development and is expressed upon activation in mature hematopoietic cells such as T and B cells, NKT cells and monocytes after prolonged antigen exposure.

Without being bound by any theory, it is contemplated that binding of the ligand PD-L1 to PD-1 downregulates effector anti-tumor T-cell activity and facilitates immune evasion. This is supported by the finding of an association between PD-1/PD-L1 expression and poor prognosis in several tumor types including gastric, ovarian, lung and renal carcinomas. PD-1 has been reported to be predominantly expressed by tumor infiltrating T lymphocytes, in melanoma.

In vitro studies of PD-1 blockade by PD-1-specific antibody showed augmentation of cytotoxic T-cell responses to melanoma-specific antigens including increased frequencies of IFN-γ-secreting antigen-specific cells.

Without being bound by any theory, it is contemplated that targeting PD-1 may act as an effective therapeutic strategy for cancer.

The principal method for targeting PD-1 clinically has been through the development of genetically engineered monoclonal antibodies that inhibit either PD-1 or PD-L1 function.

PD-L1 has also been shown to bind to B7-1 (CD80), an interaction that also suppresses T-cell proliferation and cytokine production; however, the exact relative contributions of the PD-L1: PD-1 and PD-L1:B7-1 pathways in cancer remain unclear. The PD-1-targeting agents currently in development inhibit both pathways. However, as the binding sites for PD-1 and B7-1 are adjacent but not overlapping, agents that specifically target one or the other may potentially be developed.

Cancer cells drive high expression levels of PD-L1 on their surface, allowing activation of the inhibitory PD-1 receptor on any T cells that infiltrate the tumor microenvironment, effectively switching those cells off. Indeed, upregulation of PD-L1 expression levels has been demonstrated in many different cancer types (e.g., melanoma [40%-100%], NSCLC [35%-95%], and multiple myeloma [93%]), and high levels of PD-L1 expression have been linked to poor clinical outcomes. Furthermore, tumor-infiltrating T cells have been shown to express significantly higher levels of PD-1 than T cells that infiltrate normal tissue. It is thought that the tumor microenvironment may secrete pro-inflammatory cytokines, including interferon-gamma (IFNγ) to upregulate the expression of PD-1 on tumor-infiltrating T cells to ensure that they can respond to the high levels of PD-L1 expressed on the tumor.

### MPDL3280A

MPDL3280A, also known as atezolizumab, is a human anti-PD-L1 mAb directed against the protein ligand PD-L1 (programmed cell death-1 ligand 1), with potential immune checkpoint inhibitory and antineoplastic activities. MPDL3280A contains an engineered fragment crystallizable (Fc) domain designed to optimize efficacy and safety by minimizing antibody-dependent cellular cytotoxicity (ADCC). Without being bound by any specific theory, it is understood that this structure allows inhibition of the PD-1/PD-L1 interaction, while minimizing ADCC-mediated depletion of activated T cells that is required for an effective antitumor immune response. MPDL3280A is known to bind PD-L1, blocking its binding to and activation of its receptor programmed death 1 (PD-1) expressed on activated T-cells, which may enhance the T-cell-mediated immune response to neoplasms and reverse T-cell inactivation. In addition, by binding to PD-L1, atezolizumab also prevents binding of this ligand to B7.1 expressed on activated T cells, which further enhances the T-cell-mediated immune response.

MPDL3280A has been evaluated in a phase I trial in patients with locally advanced or metastatic solid tumors. A total of 175 patients had been recruited to date. The antibody was administered as a single agent at escalating doses of ≤1, 3, 10, 15, and 20 mg/kg for a median duration of 127 days. The results of two expansion cohorts have also been reported; a cohort of 85 patients (53 of whom were evaluable for efficacy) with squamous or non-squamous non small cell lung cancer (NSCLC) and a cohort of 45 metastatic melanoma patients (35 of whom were evaluable for efficacy). In both cohorts doses of ≤1, 10, 15, and 25 mg/kg MPDL3280A were administered every 3 weeks for up to 1 year. Of the 85 patients in the NSCLC cohort, 55% were heavily pretreated with at least three prior therapies, and 81% were smokers or ex-smokers and 19% were never-smokers. The 24-week progression free survival (PFS) rate was 44% in squamous cell NSCLC and 46% in non-squamous cell NSCLC.

### Triple Negative Breast Cancer

Triple-negative breast cancer, characterized by tumors that do not express estrogen receptor (ER), progesterone receptor (PR), or Her-2 genes, represents an important clinical challenge because these cancers do not respond to endocrine therapy or other available targeted agents. The metastatic potential in triple-negative breast cancer is similar to that of other breast cancer subtypes, but these tumors are associated with a shorter median time to relapse and death. One important goal is therefore the identification of prognostic factors and markers to reliably select high and low risk subsets of patients with triple-negative disease for different treatment approaches of subtypes with differential responsiveness to specific agents. However, a reliable prognostic marker has been elusive, and markers have been inconsistently useful. For example, epidermal growth factor receptor (EGFR) has been studied, but there is still a lack of agreement on a standard assay or cutoff for EGFR expression levels with respect to prognosis. Similarly, because triple-negative status is sometimes used as a surrogate for basal-like breast cancer, specific basal markers have been explored. Indeed, trials designed to accrue patients with basal-like breast cancer using ER/PR and Her-2 negativity may provide only an approximation of the triple-negative population and are sometimes reanalyzed using more specific indicators like CK 5/6, EGFR status, and others, again marred by discordances.

Chemotherapy remains the mainstay of treatment of triple-negative breast cancer, but important limitations still need to be overcome in the next few years if any significant clinical strides are to be made. Current treatment strategies for triple-negative disease include anthracyclines, taxanes, ixabepilone, platinum agents, and biologic agents. More recently, EGFR inhibition has been proposed as a therapeutic mechanism in triple-negative breast cancer, again with mixed results. Agents that target poly(ADP-ribose) polymerase and androgen receptors have also been proposed in these patients or subsets of them, and ongoing trials should result in definitive guidance with respect to the value of these agents in triple-negative disease. Triple-negative breast cancer is clearly a distinct clinical subtype, from the perspective of both ER and Her-2 expression, but further subclassification is needed. At present, there is not a clear, proven effective single agent that targets a defining vulnerability in triple-negative breast cancer.

Various subtypes of triple negative breast cancer includes basal like TNBC (Basal like 1 and 2 (BL-1, BL-2), Immunomodulatory (IM)) and mesenchymal stem like triple negative breast cancer (MSL), and luminal androgen receptor (LAR) subtype.

PD-L1 is expressed on many cancers including renal cell carcinoma, pancreatic cancer, ovarian cancer, gastric cancer, esophageal cancer, and hepatocellular carcinoma. Research has identified the expression of PD-L1 in 50% (22 out of 44 of tumors evaluated in a breast cancer study). In 15 (34%) it was restricted to the tumor epithelium, whereas in 18 (41%) it was identified in tumor infiltrating lymphocytes. Furthermore, it was found that intratumoral expression of PD-L1 was associated with high histologic grade and negative hormone receptor status. Consistent with the previous study, it was also observed in a separate study that approximately 20% of TNBC tumors express PD-L1. The majority (95%) of these TNBC tumors were grade 3.

Without being bound by any specific theory it is hypothesized that a possible mechanism by which tumors can drive PD-L1 expression is by oncogenic signaling pathways. This was first demonstrated in glioblastomas where it was observed that PTEN loss was associated with increased PD-L1 expression, suggesting the involvement of the PI3K pathway. Because PTEN loss is commonly seen in TNBC, a study investigated the relationship between PTEN and PD-L1 expression. In approximately 50% of TNBC tumors included in the breast cancer tissue microarrays where there was >5% PD-L1 expression, a loss of PTEN staining was observed. Similarly, in a panel of TNBC cell lines, it was found that two exemplary cell lines with PTEN loss, MDA-MB-468 and BT-549, had high cell surface PD-L1 expression. Together, these data suggested that there are likely multiple mechanisms of PD-L1 regulation in TNBC.

### Methods for the Treatment of Triple Negative Breast Cancer

One embodiment provides a method of treating cancer in a patient, wherein the method comprises, administering to the patient a combination comprising entinostat and an anti-PD-L1 antibody. Another embodiment provides the method, wherein the anti PD-L1 antibody is MPDL3280A.

Another embodiment provides the method, wherein the cancer is characterized by overexpression of PD-L1. Another embodiment provides the method, wherein the cancer is triple negative breast cancer. Another embodiment provides the method, wherein the cancer is metastatic triple negative breast cancer. Another embodiment provides the method, wherein the cancer is basal like subtype of triple negative breast cancer. Another embodiment provides the method, wherein the cancer is basal like subtype-1 of metastatic triple negative breast cancer. Another embodiment provides the method, wherein the cancer is basal like subtype-2 of triple negative breast cancer. Another embodiment provides the method, wherein the cancer is immunomodulatory subtype of triple negative breast cancer. Another embodiment provides the method, wherein the cancer is mesenchymal stem cell like subtype of metastatic triple negative breast cancer. Another embodiment provides the method, wherein the cancer is basal like luminal androgen receptor subtype of triple negative breast cancer.

Another embodiment provides the method, wherein the entinostat and anti-PD-L1 antibody are administered sequentially in either order or simultaneously. Another embodiment provides the method, wherein the entinostat and anti-PD-L1 antibody are administered sequentially in either order or simultaneously, during a treatment cycle of 21 days. Another embodiment provides the method, wherein the anti-PD-L1 antibody is administered on day 1 of the treatment cycle. Another embodiment provides the method, wherein the anti-PD-L1 antibody is administered at a dose of 1200 mg. Another embodiment provides the method, wherein the anti-PD-L1 antibody is administered as intravenous infusion. Another embodiment provides the method, wherein the anti-PD-L1 antibody is administered once every two weeks at a dose of 1200 mg, by intravenous infusion. Another embodiment provides the method, wherein the entinostat is administered periodically during the treatment cycle. Another embodiment provides the method, wherein the entinostat is administered on day 1 of the treatment cycle. Another embodiment provides a method wherein the entinostat is administered orally. Another embodiment provides the method, wherein the entinostat is administered at a dose of 3 mg. Another embodiment provides the method, wherein the entinostat is administered at a dose of 5 mg. Another embodiment provides the method, wherein the entinostat is administered at a dose of 10 mg. Another embodiment provides the method, wherein the entinostat is administered orally once every week during the treatment cycle at a dose of 3 mg. Another embodiment provides the method, wherein the entinostat is administered orally once every week during the treatment cycle at a dose of 5 mg. Another embodiment provides the method, wherein the entinostat is administered orally once every two weeks during the treatment cycle at a dose of 10 mg. Another embodiment provides the method, wherein entinostat is administered first. Another embodiment provides the method, wherein entinostat is administered periodically. Another embodiment provides the method, wherein entinostat is administered weekly. Another embodiment provides the method, wherein entinostat is administered every two weeks. Another embodiment provides the method, wherein the entinostat is administered every two weeks, at a dose of 10 mg. Another embodiment provides the method, wherein entinostat and anti-PD-L1 antibody are administered simultaneously.

### Methods of Selecting Patients for Combination Therapy

Provided herein in a further embodiment is a method of selecting patients for the combination therapy comprising administration of entinostat, and an anti-PD-L1 antibody, wherein the selection is based on the level of PD-L1 expression in tumor.

Provided herein in a further embodiment is a method of selecting patients for the combination therapy comprising administration of entinostat, and an anti-PD-L1 antibody, wherein the selection is based on the tumor proportion score (TPS) for PD-L1 expression. Tumor proportion score is a measure of the percentage of cells in a tumor tissue sample that stains positive for PD-L1 expression, as determined using immuhistochemistry. In some embodiments, the TPS is determined using a PD-L1 IHC 22C3 pharmDx kit.

In some embodiments, the method further comprises administering the combination therapy comprising entinostat, and an anti-PD-L1 antibody to patients expressing elevated levels of PD-L1 in the tumor. In some embodiments, the method further comprises administering the combination therapy comprising entinostat, and an anti-PD-L1 antibody to patients wherein the TPS is between 1% and 50%. In some embodiments, the method further comprises administering the combination therapy comprising entinostat, and an anti-PD-L1 antibody to patients wherein the TPS is greater than or equal to 50%. In some embodiments, the tumor tissue sample wherein PD-L1 expression is measured is obtained from a metastatic triple negative breast cancer patient. In some embodiments, the dosage of an anti-PD-L1 antibody used in the combination therapy with entinostat is determined based on PD-L1 expression in tumor samples.

### Additional Therapy

Available additional treatments for triple negative breast cancer that may be advantageously employed in combination with the therapies disclosed herein include, without limitation, radiation therapy, chemotherapy, antibody therapy, and tyrosine kinase inhibitors as adjuvant therapy.

Radiation therapy is a cancer treatment that uses high-energy x-rays or other types of radiation to kill cancer cells or keep them from growing. Chemotherapy is a cancer treatment that uses drugs to stop the growth of cancer cells, either by killing the cells or by stopping them from dividing. When chemotherapy is taken by mouth or injected into a vein or muscle, the drugs enter the bloodstream and can reach cancer cells throughout the body (systemic chemotherapy). When chemotherapy is placed directly into the spinal column, an organ, or a body cavity such as the abdomen, the drugs mainly affect cancer cells in those areas (regional chemotherapy). The way the chemotherapy is given depends on the type and stage of the cancer being treated.

Different chemotherapeutic agents are known for treating breast cancer. Cytoxic agents used for treating breast cancer include cyclophosphamide (for example, Cytoaxn®), decetaxel (for example, Taxotere®), doxorubicin (for example, Adriamycin®), epirubicin (for example, Ellence®), methotrexate (e.g., Maxtrex®), paclitaxel (for example, Taxol®), capecitabin (for example, Xeloda®), carboplatin (for example, Paraplatin®, Paraplat®), eribulin (for example, Halaven®), 5-fluorouracil (for example, Adrucil®), gemcitabine (for example, Gemzar®), ixabepilone (for example, Ixempra®), vinorelbine (for example, Navelbine®), cisplatin (for example, Platinol®, Platinol-Aq®).

Different chemotherapeutic agents are known in the art for treating lung cancer. Cytoxic agents used for treating lung cancer include carboplatin (for example, Paraplatin®, Paraplat®), cisplatin (for example, Platinol®, Platinol-Aq®), crizotinib (for example Xalkori®), etoposide (for example Toposar®, VePesid®), etoposide Phosphate (for example Etopophos®), gemcitabine hydrochloride (for example Gemzar®), gemcitabine-cisplatin, methotrexate (for example Abitrexate®, Folex®, Folex Pfs®, Methotrexate Lpf®, Mexate®, Mexate-Aq®), paclitaxel (for example Taxol®), pemetrexed Disodium (for example Alimta®), and topotecan Hydrochloride (for example Hycamtin®)

Different agents are known in the art for treating melanoma, including aldesleukin (for example Proleukin®), dabrafenib (for example Tafinlar®), dacarbazine (for example DTIC-Dome®), recombinant Interferon Alfa-2b (for example Intron® A), Ipilimumab (for example Yervoy®), pembrolizumab (for example Keytruda®), Trametinib (for example Mekinist®), Nivolumab (for example Opdivo®), Peginterferon Alfa-2b (for example Pegintron®, Sylatron®), vemurafenib (for example Zelboraf®).

Monoclonal antibody therapy is a cancer treatment that uses antibodies made in the laboratory, from a single type of immune system cell. These antibodies can identify substances on cancer cells or normal substances that may help cancer cells grow. The antibodies attach to the substances and kill the cancer cells, block their growth, or keep them from spreading. Monoclonal antibodies are given by infusion. They may be used alone or to carry drugs, toxins, or radioactive material directly to cancer cells. Monoclonal antibodies are also used in combination with chemotherapy as adjuvant therapy.

Additional, illustrative, treatments that may be advantageously combined with the compositions and therapies disclosed herein may include, without limitation, administration of agents including, but not limited to lapatinib, alone or in combination with capecitabine, docetaxel, epirubicin, epothilone A, B or D, goserelin acetate, paclitaxel, pamidronate, bevacizumab, cetuximab or trastuzumab.

In some embodiments, the additional therapy comprises chemotherapy comprising administering to the subject one or more of doxorubicin, cyclophosphamide, paclitaxel, lapatinib, capecitabine, trastuzumab, bevacizumab, gemcitabine, eribulin, or nab-paclitaxel.

### Oral Formulations

Oral formulations containing the active pharmaceutical ingredients described herein may comprise any conventionally used oral forms, including: tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups, buccal forms, and oral liquids. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. In some embodiments are surface modifying agents which include nonionic and anionic surface modifying agents. For example, surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

### Oral Administration

As described herein, the combination therapy described herein can be given simultaneously or can be given in a staggered regimen, with entinostat being given at a different time during the course of therapy than the anti-PD-L1 antibody. This time differential may range from several minutes, hours, days, weeks, or longer between administrations of the two compounds. Therefore, the term combination does not necessarily mean administered at the same time or as a unitary dose, but that each of the components are administered during a desired treatment period. The agents may also be administered by different routes.

In other embodiments, the pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

In further embodiments, the pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

In other embodiments, the pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfate, sodium metabisulfite, thiodipropionic acid and its esters, and dithiocarbamates.

The pharmaceutical compositions provided herein for oral administration may be also provided in the forms of liposomes, micelles, microspheres, or nanosystems. Miccellar dosage forms can be prepared as described in U.S. Pat. No. 6,350,458.

In other embodiments, the pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

In further embodiments, the pharmaceutical compositions provided herein may be co-formulated with other active ingredients which do not impair the desired therapeutic action, or with substances that supplement the desired action.

### EXAMPLES

### Example 1

A Phase 1B/2, open-label, dose escalation study of entinostat in combination with MPDL3280A in patients with metastatic triple negative breast cancer.

Entinostat has been shown in preclinical models to reduce the number of, and inhibit the function of, host immune suppressor cells in order to enhance the anti-tumor activity of immune checkpoint blockade. It is hypothesized that entinostat combined with MPDL3280A will result in an improved response rate for the combination compared to either agent alone. Preclinical study data suggest that entinostat specifically targets MDSCs and thus improves the response to PD-L1-blocking antibody (i.e., MPDL3280A) treatment. The study evaluates populations of MDSCs and other myeloid cells in peripheral blood and tumor tissues as well as basic T-cell function in patients, with the expectation that if the MDSC level is decreased, the response to antigens improved.

Phase 1/B (Dose Escalation Phase)

### Primary Objective:

Determine the dose-limiting toxicities (DLT) and maximum tolerated dose (MTD) or recommended Phase 2 dose (RP2D) of entinostat (SNDX-275) given in combination with MPDL3280A.

### Secondary Objectives:

### Safety

Evaluate safety and the tolerability of entinostat in combination with MPDL3280A, as measured by clinical adverse events (AEs) and laboratory parameters.

### Efficacy

Evaluate the efficacy of entinostat in combination with MPDL3280A in patients with mTNBC, as determined by secondary measures of efficacy, including:
Clinical benefit rate (CBR) (i.e. complete response [CR]+partial response [PR]+stable disease [SD]) at 6 months.
Progression-free survival (PFS) status at 6 months.
PFS
Overall survival (OS)

In patients who experience a response to treatment (i.e., CR or PR):
Duration of response (DOR)
Time to response (TTR)

### Exploratory

Following are the exploratory objectives for the study:
Evaluate changes in expression of immune checkpoint receptors/ligands (programmed death receptor-1 [PD-1]/programmed death ligand-1 [PD-L1]) in tumor biopsies pre- and post-therapy.
Assess the ratio of effector T cells: regulatory T cells in blood and tumor biopsies pre- and post-therapy.
Evaluate inflammatory T cell signature changes in blood and tumor biopsies pre- and post-therapy.
Evaluate changes in number of myeloid-derived suppressor cells (MDSCs) in peripheral blood and tumor biopsies pre- and post-therapy.
Evaluate changes in protein lysine acetylation in peripheral blood cells and tumor biopsies pre- and post-therapy.

### Study Design:

The study is an open-label, Phase 1b/2 study evaluating the combination of entinostat plus MPDL3280A in patients with metastatic triple negative breast cancer (mTNBC). The study has 2 phases: a Dose Escalation/Confirmation Phase (Phase 1b) and an Expansion Phase (Phase 2), with the Expansion Phase utilizing a Simon 2-stage design for each cohort.

For both phases, patients are screened for study eligibility within 21 days before the first study drug dose. Patients who are determined to be eligible, based on screening assessments are enrolled in the study on Cycle 1, Day 1 (C1D1; baseline) and receive entinostat in combination with MPDL3280A.

A cycle is 21 days in length. During treatment, patients attend study center visits and have study evaluations performed on C1D1, C1D8, and C1D15; D1 and D15 of C2; and on D1 of each cycle thereafter.

The starting dose (dose level 1) for entinostat is 5 mg by mouth (po) weekly. The dose of MPDL3280A is fixed at 1200 mg IV every three weeks (q 3 weeks) for all cohorts.

If dose level 1 is not tolerated, dose level −1 for entinostat is set at 3 mg po weekly. If dose level 1 is tolerated, dose level 2, of 10 mg by mouth (po) once every two weeks (Q2W) is explored.

Each dose level in the dose escalation phase enrolls a maximum of 6 evaluable patients. Therefore a maximum of 12 evaluable patients are enrolled in the dose escalation phase.

TABLE 1
Dose Escalation Schematic

| | Number of | | | MPDL3280A Dose |
|---|---|---|---|---|
| Cohort | Subjects | Entinostat Dose | | Q3W |
| −1 | 2-6 | 3 | mg po QW | 1200 mg IV infusion |
| 1 | 2-6 | 5 | mg po QW | 1200 mg IV infusion |
| 2 | 2-6 | 10 | mg po Q2W | 1200 mg IV infusion |

### Collection of Tumor Tissue and Blood Samples

Fresh tumor tissue samples are collected during the study as follows during screening from all patients on a mandatory basis.

Archival biopsies when available are collected for comparison.

Further, tumor tissue samples are also optionally collected on C2D15 (±3 days) from patients in the Dose Escalation/Confirmation Phase. All patients in the Dose Escalation/Confirmation Phase are strongly encouraged to provide an optional biopsy in order to help understand dose-immune correlate effects.

Tumor tissue samples are collected on C2D15 (±3 days) on a mandatory basis from the first 10 patients in Stage 1 in the Expansion Cohort.

If, based on an interim review of tumor tissue data from the initial patients in the Expansion Phase, such data are considered informative, then tumor tissue samples are collected on a mandatory basis from all subsequent patients in the Expansion Phase on C2D15 (+3 days). Alternatively, if such data are not considered informative, these samples are collected from subsequent patients.

Blood for immune correlates is collected pre-dose on C1D1, C2D1, C2D15 and C3D1. Samples are also collected on C2D15 for pharmacokinetic (PK) analysis.

### Radiological Assessment

Patients are radiologically assessed during screening and every 6 weeks (±3 days) (Week 6, Week 12, etc.) to assess disease progression. Disease is assessed by computed tomography (CT), magnetic resonance imaging (MRI), and bone scans, as appropriate, and response to the combination therapy is assessed by the Investigator, primarily using RECIST 1.1.

### Safety

Safety is assessed during the study by documentation of AEs, clinical laboratory tests, physical examination, vital sign measurements, electrocardiograms (ECGs), and Eastern Cooperative Oncology Group (ECOG) performance status.

### Duration of Treatment

The maximum duration of treatment for this study is planned to be 2 years. If a patient permanently discontinues one of the two study drugs (either entinostat or MPDL3280A), the patient may continue to receive monotherapy for 2 years, unless alternate therapy is started or another discontinuation criterion is meet. After discontinuation of both study drugs, patients complete an End of Treatment (EOT) visit within 7 days after the last study drug dose and a Safety Follow-up (F/U) visit 30 days thereafter. After completion of the 30-day Safety F/U visit, patients who have not experienced progressive disease (PD) are followed every 2 months until PD and every 3 months thereafter until death or closure of the study.

### Phase 1b (Dose Escalation/Confirmation)

The Dose Escalation/Confirmation Phase of the study, in which patients with metastatic TNBC (mTNBC) are enrolled, employs a classical 3+3 design, with the determination of DLT and the MTD and/or RP2D based on entinostat in combination with MPDL3280A in cycle 1 (C1).

Although decisions regarding dose escalation are made primarily based on review of data from C1, safety data is also collected from all patients continuing treatment and reviewed in an ongoing manner by the Medical Monitor in consultation with the Investigators. Any detected cumulative toxicity may require later dose reductions and/or other changes to the dosing schedule, as appropriate, including further refinement of the RP2D.

### Dose Escalation

The initial 3-6 patients receive entinostat at a starting dose of 5 mg on D1, D8, and D15 along with MPDL3280A at a dose of 1200 mg, via intravenous infusion, on D1 of a 21-day cycle.

If the safety profile of the 5 mg dosage is acceptable, evaluation of an alternate schedule of entinostat dose 10 mg once every two weeks (Q2W) is administered, keeping the total MPDL3280A dose exposure constant. However, based on evaluation of the safety and tolerability data of the previous dose level, it may also be decided that accrual takes place at an intermediate dose level or an alternate dosing schedule.

If the 5 mg dose exceeds the MTD, then a 3 mg dose is evaluated. Toxicities are assessed by the study Investigator using the United States (US) National Cancer Institute (NCI) Common Terminology Criteria for Adverse Events (CTCAE), version 4.03. The decision regarding whether to proceed to the next dose level is made by the Medical Monitor in consultation with the study Investigators after the majority of the safety assessments for each cohort are completed.

All patients within a cohort are to complete C1, have safety assessments performed through C2D1, and be assessed for DLT before enrollment of the next cohort may commence. A maximum tolerated dose (MTD) is selected based on the extent of DLT experienced by patients within a cohort.

### Dose Confirmation

The prospective MTD/RP2D(s) identified in the Dose Escalation Phase is confirmed in at least 9 patients in Dose Confirmation Cohort(s) to obtain additional AE, immune correlate, and anti-tumor activity data on entinostat in combination with MPDL3280A. In the event that both the 5 mg once every week (QW) and 10 mg once every two weeks (Q2W) do not exceed the MTD, both dose levels are confirmed in parallel.

After completion of the Dose Escalation/Confirmation Phase of the study, with identification of the MTD/RP2D, the Phase 2 portion of the study commences.

Phase 2 (Expansion): In the Expansion Phase, entinostat in combination with MPDL3280A is evaluated using the RP2D identified in the Dose Escalation/Confirmation Phase in mTNBC. Additional Expansion Cohorts consisting of distinct subsets of patients with solid tumor cancers may be explored. Each Expansion Cohort evaluated during the Expansion Phase employs a Simon 2-stage design. The final decision about which Expansion Cohorts to study will be based on data from the Dose Escalation/Confirmation Phase, emerging clinical data from other studies, and/or nonclinical data.

### Sample Size Considerations:

### Dose Escalation/Confirmation Phase

A total of 3 to 6 patients are enrolled in each dose cohort based on a standard Phase 1 dose escalation scheme. Each patient participates in only 1 dose cohort. The total number of patients to be enrolled in the Dose Escalation/Confirmation Phase is dependent upon the observed safety profile, which determines the number of patients per dose cohort, as well as the number of dose escalations required to achieve the MTD or RP2D.

A starting sample size of at least 3 patients per dose cohort, expanding to 6 patients in the event of a marginal DLT rate is deemed to be a safe and conventional approach in the dose escalation of a novel oncologic agent.

At least 9 and up to 18 additional patients are enrolled at the potential RP2D in the Dose Confirmation Cohort(s) to obtain additional AE, immune correlates, and anti-tumor activity data on entinostat at the MTD or other dose recommended for further investigation in Phase 2 (i.e., RP2D) in combination.

If the first proposed RP2D is not tolerable in the Dose Confirmation cohort, a second Dose Confirmation cohort is enrolled at the lower proposed RP2D.

### Expansion Phase

In the Expansion Phase of the study, the safety and preliminary antitumor activity of entinostat, when administered at the RP2D with MPDL3280A, is explored in a cohort of up to 39 patients with mTNBC. Patients are enrolled according to a single-arm study design with ORR as the primary endpoint. The Expansion Phase is carried out in 2 stages so that enrollment for the cohort can terminate early in the event the antitumor activity of the combination regimen is not sufficient. The number of patients evaluated in each stage and the minimum number of responders needed to continue to the next stage, as described below, are determined based on the optimum version of Simon's 2-stage design. The protocol may be amended to allow for enrollment of additional or different cohorts, for example, patients with triple negative breast cancer or PD-L1-positive colorectal cancer, based on emerging data during study.

Phase 2 mTNBC Cohort

A maximum of 39 patients with mTNBC are enrolled. A true ORR of 30% is hypothesized. An ORR greater than 15% is considered a lower threshold for antitumor activity and warrant continued development.

Based on the design elements specified above, up to 19 patients with mTNBC may be enrolled during the first stage: If 3 or fewer patients achieve an objective response (CR or PR), confirmed or unconfirmed, then enrollment terminates; otherwise, 20 additional patients are enrolled during the second stage.

### Endpoints:

### Primary Efficacy Endpoint

ORR, as determined by RECIST 1.1
Secondary Endpoints: (analyzed in the same populations as the primary endpoint)
CBR (CR+PR+SD) at 6 months
PFS status at 6 months
PFS
OS
   In patients who experience a response to treatment (CR or PR):
DOR
TTR

An analysis of efficacy endpoints is also performed, with response determined using RECIST, version 1.1.

### Safety

AEs, clinical laboratory parameters, and ECGs.

### Exploratory:

Changes in expression of checkpoint inhibitors (PD-1/PD-L1) in tumor biopsies pre- and post-therapy

Ratio of effector T cells: regulatory T cells in tumor biopsies pre- and post-therapy (immunohistochemistry)

Changes in number of MDSCs in peripheral blood and tumor biopsies (flow cytometry)

Changes in protein lysine acetylation in peripheral blood cells and tumor biopsies pre- and post-therapy

### Summary of Patient Eligibility Criteria:

### Inclusion Criteria

Patients meeting all of the following criteria are considered eligible to participate in the study:
1. Has histologically or pathologically confirmed recurrent or metastatic TNBC amenable to biopsy at baseline and (for certain cohorts, as described in protocol) at least once on study.
2. Aged 18 years or older on the day written informed consent is given.
3. If has brain metastases, must have stable neurologic status following local therapy (surgery or radiation) for at least 2 weeks without the use of steroids or on stable or decreasing dose of ≤10 mg daily prednisone (or equivalent), and must be without neurologic dysfunction that would confound the evaluation of neurologic and other AEs. (Patients with a history of carcinomatous meningitis are not eligible.)
4. Evidence of locally recurrent or metastatic disease based on imaging studies (e.g., CT, MRI) within 28 days before the first study drug dose.
5. At least 1 measurable lesion ≥20 mm by conventional techniques or ≥10 mm by spiral CT scan or MRI, with the last imaging performed within 28 days before the first study drug dose. If there is only 1 measurable lesion and it is located in previously irradiated field, it must have demonstrated progression according to RECIST, version 1.1.
6. If receiving radiation therapy has a 2-week washout period following completion of the treatment prior to receiving the first study drug dose and continues to have at least 1 measurable lesion, per above criterion.
7. ECOG performance status of 0 or 1.
8. Has the following laboratory parameters:

TABLE 2
Laboratory parameters for inclusion in combination therapy

| | |
|---|---|
| System | Laboratory Value |

Hematological

| | |
|---|---|
| Absolute neutrophil | ≥1.5 × 10⁹/L |
| count (ANC) | |
| Platelets | ≥100 × 10⁹/L |
| Hemoglobin | ≥9 g/dL or ≥5.6 mmol/L |

Renal

| | |
|---|---|
| Creatinine OR | ≤1.5 × the upper limit of |
| Measured or calculated¹ | normal (ULN) OR |
| creatinine clearance | ≥60 mL/min for |
| (CrCl) (glomerular | patient with creatinine |
| filtration rate [GFR] can | levels >1.5 × institutional ULN |
| also be used in place | |
| of creatinine or CrCl) | |

Hepatic

| | |
|---|---|
| Total bilirubin | ≤1.5 × ULN OR |
| | Direct bilirubin ≤ULN for patients |
| | with total bilirubin levels >1.5 × ULN |
| Aspartate aminotransferase | ≤2.5 × ULN OR |
| (AST) and alanine | ≤5 × ULN for patients with liver |
| aminotransferase (ALT) | metastases |

Coagulation

| | |
|---|---|
| International Normalized | ≤1.5 × ULN unless patient is |
| Ratio (INR) or | receiving anticoagulant therapy as |
| Prothrombin Time (PT) | long as PT or PTT is within |
| Activated Partial | therapeutic range of intended use |
| Thromboplastin | of anticoagulants |
| Time (aPTT) | ≤1.5 × ULN unless patient is |
| | receiving anticoagulant therapy as |
| | long as PT or PTT is within |
| | therapeutic range of intended use |
| | of anticoagulants |

¹Creatinine clearance is calculated per institutional standard.

9. If a female of childbearing potential, has a negative blood pregnancy test within 72 hours prior to receiving the first dose of study drug. A urine test can be considered if a blood test is not appropriate.
10. If a female of childbearing potential, willing to use 2 methods of birth control or is surgically sterile, or willing to abstain from heterosexual activity for the course of the study through 120 days after the last dose of study drug. Patients of childbearing potential are those who have not been surgically sterilized or have not been free from menses for >1 year.
11. Experienced resolution of toxic effect(s) of the most recent prior chemotherapy to Grade 1 or less (except alopecia or neuropathy). If patient underwent major surgery or radiation therapy of >30 Gy, they must have recovered from the toxicity and/or complications from the intervention.
12. Able to understand and give written informed consent and comply with study procedures.

### Exclusion Criteria

Patients meeting any of the following criteria are not eligible to participate in the study:
1. Diagnosis of immunodeficiency or receiving systemic steroid therapy or any other form of immunosuppressive therapy within 7 days prior to the first dose of study drug. The use of physiologic doses of corticosteroids may be approved after consultation with the Sponsor.
2. Active autoimmune disease including active diverticulitis, symptomatic peptic ulcer disease, colitis, or inflammatory bowel disease that has required systemic treatment in past 2 years (i.e., with disease modifying agents, corticosteroids, or immunosuppressive drugs). Replacement therapy (e.g., thyroxine, insulin, or physiologic corticosteroid replacement therapy for adrenal or pituitary insufficiency) is not considered a form of systemic treatment.
3. Allergy to benzamide or inactive components of entinostat.
4. History of allergies to any active or inactive ingredients of MPDL3280A.
5. History or current evidence of any condition, therapy, or laboratory abnormality that might confound the results of the study, interfere with the patient's participation for the full duration of the study, or is not in the best interest of the patient to participate, in the opinion of the treating Investigator, including, but not limited to:
   Myocardial infarction or arterial thromboembolic events within 6 months prior to baseline or severe or unstable angina, New York Heart Association (NYHA) Class III or IV disease, or a QTc interval >470 msec.
   Uncontrolled heart failure or hypertension, uncontrolled diabetes mellitus, or uncontrolled systemic infection.
   Another known additional malignancy that is progressing or requires active treatment (excluding adequately treated basal cell carcinoma or cervical intraepithelial neoplasia [CIN]/cervical carcinoma in situ or melanoma in situ). Prior history of other cancer is allowed, as long as there is no active disease within the prior 5 years.
   Active infection requiring systemic therapy.
   Known active central nervous system (CNS) metastases and/or carcinomatous meningitis.
6. Known psychiatric or substance abuse disorders that would interfere with cooperation with the requirements of the study.
7. Currently participating and receiving study therapy or has participated in a study of an investigational agent and received study therapy or used an investigational device within 4 weeks of the first dose of treatment.
8. Received a live vaccine within 30 days of the first dose of treatment.
9. Prior anti-cancer monoclonal antibody (mAb) within 4 weeks prior to baseline or who has not recovered (i.e., Grade 1 or at baseline) from AEs due to agents administered more than 4 weeks earlier.
10. Prior chemotherapy, targeted small molecule therapy, or radiation therapy within 2 weeks prior to study baseline or who has not recovered (i.e., ≤Grade 1 or at baseline) from AEs due to a previously administered agent. Patients with ≤Grade 2 neuropathy or ≤Grade 2 alopecia are an exception to this criterion and may qualify for the study. If patient underwent major surgery, they must have recovered adequately from the toxicity and/or complications from the intervention prior to starting therapy.
11. Received transfusion of blood products (including platelets or red blood cells) or administration of colony stimulating factors (including granulocyte-colony stimulating factor [G-CSF], granulocyte macrophage-colony stimulating factor [GM-CSF], or recombinant erythropoietin) within 4 weeks prior to the first dose of treatment.
12. Currently receiving treatment with any other agent listed on the prohibited medication list such as valproic acid, or other systemic cancer agents within 14 days of the first dose of treatment.
13. If female, is pregnant, breastfeeding, or expecting to conceive, or if male, expect to father children within the projected duration of the study, starting with the screening visit through 120 days after the last dose of study drug.
14. Known history of human immunodeficiency virus (HIV) (HIV 1/2 antibodies).
15. Known active hepatitis B (e.g., hepatitis B surface antigen-reactive) or hepatitis C (e.g., hepatitis C virus ribonucleic acid [qualitative]).
16. Is or has an immediate family member (e.g., spouse, parent/legal guardian, sibling, or child) who is investigational site or sponsor staff directly involved with this study, unless prospective Institutional Review Board (IRB)/Ethics Committee (EC) approval (by chair or designee) is given allowing exception to this criterion for a specific patient.
17. Previously treated with a PD-1/PD-L1-blocking antibody (e.g., MPDL3280A, nivolumab, pembrolizumab) or a histone deacetylase inhibitor (e.g., vorinostat, belinostat, romidepsin, panobinostat).
18. Patients with previously treated brain metastases may participate provided they are stable (without evidence of progression by imaging [using the identical imaging modality for each assessment, either MRI or CT scan] for at least 4 weeks prior to the first dose of study drug and any neurologic symptoms have returned to baseline), have no evidence of new or enlarging brain metastases, and are not using steroids for at least 7 days prior to the first dose of study drug. This exception does not include carcinomatous meningitis which is excluded regardless of clinical stability.

### Statistical Considerations

The safety and efficacy analyses are presented by study phase. For the escalation phase, tabulations are provided by dose cohort and overall. For the Expansion Phase, tabulations are provided by tumor type and overall. Some analyses are performed based on the Dose Escalation/Confirmation and Expansion Phases combined.

### Safety Analysis

Treatment-emergent AEs reported during the study are tabulated and listed by Medical Dictionary for Regulatory Activities (MedDRA) System Organ Class (SOC) and Preferred Term (PT). Tables display number and percentage of patients experiencing the event for the following categories: all AEs; AEs considered related to study drug; AEs by severity; DLTs; AEs occasioning treatment delay or discontinuation; and serious adverse events (SAEs).

For the Dose Escalation/Confirmation Phase, the observed DLT rate in each dose cohort is calculated by the crude proportion of patients who experienced DLT with a 2-sided 95% exact binomial confidence interval (CI).

Hematology and serum chemistries are summarized using conventional summary statistics (mean, standard deviation, median, and range) for the following: baseline value, minimum and maximum post baseline values, average post baseline value, and last post baseline value. Standard shift tables will also be prepared presenting worst post baseline toxicity grade versus baseline. Vital signs are summarized in a descriptive manner by calculating the mean, standard deviation, median, and range in the same manner described for laboratory values. The Wilcoxon signed rank test may be used to assist in the identification of any systematic changes.

### Efficacy Analyses

Efficacy analyses are conducted using the Full Analysis Set and, where appropriate, the Per-protocol set. ORR is estimated for each cohort evaluated in the Expansion Phase, assessed using RECIST 1.1. Crude proportion of patients with best overall response of CR or PR, along with a 2-sided 95% CI, is calculated. The width of the CI is adjusted to account for the multistage design. Additionally, a 90% one-sided CI of the form (πL, 1] is reported since the sample size for the Expansion Phase is determined using a one-sided significance level of 10%. CBR at 6 months is analyzed in a similar manner.

DOR is calculated for patients who achieve a CR or PR and is defined as the number of months from the start date of the response (and subsequently confirmed) to the first date that recurrent disease or PD is documented. PFS is defined as the number of months from the date of the first dose of study drug to the earliest of documented PD or death due to any cause without prior progression. OS is defined as the number of months from the first dose of study drug to the date of death due to any cause. DOR, PFS, and OS is summarized descriptively using the Kaplan-Meier method with 95% CIs calculated using Greenwood's formula. Median follow-up for each endpoint is estimated according to the Kaplan-Meier estimate of potential follow-up. PFS rate at 6 months and corresponding 95% CIs are estimated using the Kaplan-Meier method. Greenwood's formula is used to calculate the standard errors of the Kaplan-Meier estimate and upper and lower limits of the 95% CI.

### Procedures:

The schedule of study is listed in Table 3

TABLE 3
Schedule of Study Assessments

| | Screening | Combination Therapy | | | | | | | | Post- |
|---|---|---|---|---|---|---|---|---|---|---|
| | (D −21 | C1 | | | C2 | | ≥C3 | | Safety | Study |
| Procedure | to −1) | D1 | D8 | D15 | D1 | D15 | D1 | EOT¹ | F/U² | F/U³ |
| Visit Window | — | — | ±1D | ±1D | ±3D | ±3D | ±3D | ±3D | ±5D | ±7D |
| Provision of | X | | | | | | | | | |
| written | | | | | | | | | | |
| informed | | | | | | | | | | |
| consent | | | | | | | | | | |
| Demographics | X | | | | | | | | | |
| Height | X | | | | | | | | | |
| Medical | X | X | | | | | | | | |
| history, | | | | | | | | | | |
| including | | | | | | | | | | |
| underlying | | | | | | | | | | |
| disease history | | | | | | | | | | |
| Complete | X⁴ | X⁴ | | | | | | X | X | |
| physical | | | | | | | | | | |
| examination | | | | | | | | | | |
| Symptom- | | X | X | X | X | X | X | | | |
| directed | | | | | | | | | | |
| physical | | | | | | | | | | |
| examination | | | | | | | | | | |
| ECG | X | | | | | | X⁵ | X | | |
| Vital signs and | X | X | | | X | | X | X | X | |
| weight | | | | | | | | | | |
| ECOG | X | X | | | X | | X | X | X | |
| performance | | | | | | | | | | |
| status | | | | | | | | | | |
| Radiological | X⁶ | | | | | | X⁷ | X⁸ | | |
| & Physical | | | | | | | | | | |
| Disease | | | | | | | | | | |
| Response | | | | | | | | | | |
| Assessment | | | | | | | | | | |
| Pregnancy | X | X⁹ | | | | | | | | |
| testing | | | | | | | | | | |
| Hematology, | X | X¹² | | X | X | X | X | X | | |
| coagulation | | | | | | | | | | |
| studies¹⁰, and | | | | | | | | | | |
| clinical | | | | | | | | | | |
| chemistries¹¹ | | | | | | | | | | |
| Blood sample | | X | | | X | X | X¹³ | | | |
| for immune | | | | | | | | | | |
| correlates | | | | | | | | | | |
| Blood sample | | X | | X | | | | | | |
| for protein | | | | | | | | | | |
| lysine | | | | | | | | | | |
| acetylation | | | | | | | | | | |
| Tissue sample | X | | | | | X¹⁴ | | X | | |
| collection for | | | | | | | | | | |
| immune | | | | | | | | | | |
| correlates | | | | | | | | | | |
| Entinostat | | Entinostat is to be self-administered by the | | | | | | | | |
| self- | | patient weekly (or biweekly if on a Q2W | | | | | | | | |
| administration | | schedule), starting on C1D1 | | | | | | | | |
| MPDL3280A | | X | | | X | | X | | | |
| administration | | | | | | | | | | |
| Pre- | X | X | X | X | X | X | X | X | X | |
| treatment/concomitant | | | | | | | | | | |
| medications | | | | | | | | | | |
| Adverse | X | X | X | X | X | X | X | X | X | |
| events | | | | | | | | | | |
| Study drug | | X | X | X | | | | X | | |
| compliance | | | | | | | | | | |
| assessment | | | | | | | | | | |

| | |
|---|---|
| Post-study | X |
| treatment | |
| patient | |
| contact¹⁵ | |

Table Legend:
1. The EOT visit is conducted within 7 days of study drug discontinuation.
2. The first Safety F/U visit is 30 days after the EOT visit. After completion of the Safety F/U visit, patients who have not experienced PD are followed every 2 months until PD and 3 months thereafter until death or closure of the study by the Sponsor.
3. After completion of the Safety F/U visit, patients who have not experienced PD are followed every 2 months until PD and 3 months thereafter until death or closure of the study by the Sponsor.
4. If the screening physical examination is performed within 7 days before baseline (C1D1), then a symptom-directed examination may be performed at baseline.
5. An ECG is to be performed pre-dose on C3D1 and then every 3 cycles thereafter. An ECG may be repeated anytime, as clinically indicated.
6. Performed only if last scan is performed more than 28 days previously.
7. Patients have radiological disease assessments performed during screening and every 6 weeks (±3 days) (Week 6, Week 12, Week 18, Week 24, etc.) during treatment and, for patients who have not yet progressed, until progression. For patients with negative CT findings at baseline, a CT is required only if clinically indicated.
8. Performed only if radiological progression was not previously observed on study.
9. For female patients of child-bearing potential, a serum pregnancy test is performed during screening and within 72 hours before the first study drug dose. Pregnancy testing is to be repeated during the study any time pregnancy is suspected.
10. Analytes tested include white blood cell count (WBC) with absolute counts of individual cell types, platelet count, hemoglobin (HGB), and, at screening only, prothrombin time (PT) or international normalized ratio (INR) and activated partial thromboplastin time (aPTT).
11. Analytes tested include ALT, AST, albumin, alkaline phosphatase, total bilirubin, blood urea nitrogen (BUN), calcium, creatinine, sodium, potassium, chloride, bicarbonate, glucose, lactate dehydrogenase (LDH), phosphorus, total protein, and uric acid. Magnesium is measured at baseline only, unless clinically indicated.
12. Performed only if screening laboratory tests performed >7 days prior to C1D1 (baseline).
13. C3 only.
14. Fresh tumor tissue samples are collected during the study as follows: during screening from all patients on a mandatory basis; on C2D15 (±3 days) on an optional basis from patients in the Dose Escalation/Confirmation Phase; and on C2D15 (±3 days) on a mandatory basis from the first 10 patients in Stage 1 in the Expansion Phase. If, based on an interim review of tumor tissue data from the initial patients in the Expansion Phase, such data are considered informative, and then tumor tissue samples are collected on a mandatory basis from all subsequent patients in the Expansion Phase on C2D15 (±3 days). Alternatively, if such data are not considered informative, these samples are not collected from subsequent patients. Optional biopsy can be done at disease progression at the end of C4 or subsequent timepoints. Entinostat PK sample is be collected at time of biopsy once therapy has begun.
15. Information regarding PD, alternate treatments, and survival status are collected until study closure; every 2 months until PD and then every 3 months thereafter.

1. A method of treating cancer, in a patient in need thereof wherein the method comprises, administering to the patient a combination comprising entinostat and an anti-PD-L1 antibody.

2. The method of claim 1, wherein the anti PD-L1 antibody is MPDL3280A.

3. The method of claim 1, wherein the cancer is characterized by overexpression of PD-L1.

4. The method of claim 1, wherein the cancer is breast cancer.

5. The method of claim 4, wherein the breast cancer is estrogen receptor (ER), progesterone receptor (PR), and Her-2 negative breast cancer.

6. The method of claim 5, wherein the estrogen receptor (ER), progesterone receptor (PR), and Her-2 negative breast cancer is triple negative breast cancer.

7. The method of claim 6, wherein the triple negative breast cancer is metastatic triple negative breast cancer.

8. The method of claim 1, wherein entinostat and the anti-PD-L1 antibody are administered sequentially in either order or simultaneously during a treatment cycle of 21 days.

9. The method of claim 8, wherein the anti-PD-L1 antibody is administered by intravenous infusion.

10. The method of claim 9, wherein the anti-PD-L1 antibody is administered once every three weeks during the treatment cycle, at a dose of 1200 mg.

11. The method of claim 8, wherein the entinostat is administered orally.

12. The method of claim 11, wherein the entinostat is administered once every week during the treatment cycle, at a dose of 3 mg.

13. The method of claim 11, wherein the entinostat is administered once every week during the treatment cycle, at a dose of 5 mg.

14. The method of claim 11, wherein the entinostat is administered once every two weeks during the treatment cycle, at a dose of 10 mg.

15. The method of claim 1, wherein entinostat is administered first.

16. The method of claim 1, wherein the entinostat is administered weekly.

17. The method of claim 1, wherein the entinostat is administered every two weeks.

18. The method of claim 17, wherein the entinostat is administered at a dose of 10 mg.

19. The method of claim 1, wherein entinostat and anti-PD-L1 antibody are administered simultaneously.

20. A kit for treating triple negative breast cancer comprising a combination of entinostat and an anti-PD-L1 antibody.

21. The kit of claim 20, wherein the anti-PD-L1 antibody is MPDL3280A.

22. The method of claim 1, comprising administering to the patient a combination comprising entinostat and MPDL3280A, wherein the cancer is metastatic triple negative breast cancer.

23. A method of treating cancer in a patient in need thereof, wherein the method comprises administering to the patient a combination therapy comprising entinostat and an anti-PD-L1 antibody, wherein the cancer is metastatic triple negative breast cancer and the anti-PD-L1 antibody is MPDL3280A.

24. A method of treating a cancer in a patient in need thereof, wherein the method comprises, administering to the patient a combination consisting essentially of entinostat and MPDL3280A.

25. The method of claim 24, wherein the cancer is metastatic triple negative breast cancer.

26. The method of claim 23, wherein the entinostat is administered as a solid dosage form and the MPDL3280A is administered as an intravenous infusion.

27. A method of selecting a patient for a combination therapy comprising administering entinostat and an anti-PD-L1 antibody, the method comprising measuring PD-L1 expression in a tumor tissue sample obtained from the patient.

28. The method of claim 27, further comprising administering the combination therapy to the patient if tumor proportion score (TPS) for PD-L1 expression is between 1% and 50%.

29. The method of claim 27, further comprising administering to the patient the combination therapy if tumor proportion score (TPS) for PD-L1 expression is greater than or equal to 1%.

30. The method of claim 27, further comprising administering to the patient the combination therapy if tumor proportion score (TPS) for PD-L1 expression is greater than or equal to 49%.

31. The method of claim 23, wherein the tumor tissue sample is from a metastatic triple negative breast cancer.
